(19) United States
(12) Patent Application Publication
(10) Pub. No.: US 2026/0315232 A1
(43) Pub. Date: Oct. 15, 2026

(54) Magnetic Retention Device for Osseo-integrated Digit Prostheses

(71) Applicant: THE JOHNS HOPKINS UNIVERSITY, Baltimore, MD (US)

(72) Inventor: Andrew Jackson Etheridge, Baltimore, MD (US)

(21) Appl. No.: 19/647,248

(22) Filed: Apr. 14, 2026

Related U.S. Application Data
(60) Provisional application No. 63/788,459, filed on Apr. 14, 2025.

(51) Int. Cl. A61F 2/30; A61F 2/42

(52) U.S. Cl. CPC A61F 2/30749; A61F 2/4225; A61F 2/4241

(57) ABSTRACT

Designed to offer patients with an alternative option for prosthetic restoration, the OI prosthetic of the present invention leverages magnetic retention to provide ease of donning/doffing, ease of daily hygiene, the ability to use a PT/OT program for weight training the bone, and the ability to interchange various devices for task specific applications (aesthetic finger, work finger/device, hobby device, etc.). This will provide more options for OI care teams to offer improved treatment to this patient population. Individuals that would utilize this product would include prosthetist/orthotist, Clinical anaplastologist, and PM&R physicians, orthopedic surgeons, and PT/OTs.

### CROSS REFERENCE TO RELATED APPLICATIONS

This application claims the benefit of U.S. Provisional Patent Application No. 63/788,459 filed on Apr. 14, 2025, which is incorporated by reference, herein, in its entirety.

### FIELD OF THE INVENTION

The present invention relates generally to prosthetic devices. More particularly, the present invention relates to a magnetic retention device for osseo-integrated digital prosthesis.

### BACKGROUND OF THE INVENTION

Prosthetic digits for finger and toes are restorative devices that provide aesthetic and functional rehabilitation. These devices are intended to be removed daily for hygienic purposes. OI (osseo-integrated) implants are in common use for dental, facial, and trans-femoral applications. However, such OI implants are not available for other implementations, and therefore have also not been optimized for use in other applications.

It would therefore be advantageous to provide a magnetic retention device for osseo-integrated digital prosthesis.

### SUMMARY OF THE INVENTION

The foregoing needs are met, to a great extent, by the present invention directed to a device including an osseo-integrated fixture, wherein the osseo-integrated fixture has a proximal end and a distal end. The proximal end is configured to be integrated into a bone, and the distal end includes a magnet. The device also includes an attachment component. The attachment component is configured to connect magnetically with the osseo-integrated fixture.

In accordance with an aspect of the present invention, the device is configured to provide an osseo-integrated toe. The attachment further includes an aesthetic toe. The device is configured to provide an osseo-integrated finger. The attachment component can include one selected from a group consisting of an aesthetic finger, a work finger, work device, hobby device, and a physical therapy device.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings like reference numerals identify corresponding elements and:

FIGS. 1A-1E illustrate perspective and partially exploded views of retention systems and abutments for an OI prosthesis, according to an embodiment of the present invention.

FIGS. 2A-2C illustrate side views of a retention system and abutment for an OI prosthesis, according to an embodiment of the present invention.

FIGS. 3A-3F illustrate perspective and partially exploded views of retention systems and abutments for an OI prosthesis, according to an embodiment of the present invention.

FIGS. 4A-4F illustrate perspective views of a magnetic cap insertion according to an embodiment of the present invention.

FIGS. 5A-5E illustrate perspective and partially exploded views of retention systems and abutments for an OI prosthesis, according to an embodiment of the present invention.

FIGS. 6A-6C illustrate perspective views of a device according to the present invention mounted on an osseo-integrated base.

FIGS. 7A-7C illustrate perspective and semi-sectional views of an abutment and retention system, according to an embodiment of the present invention.

FIGS. 8A and 8B illustrate perspective views of an abutment and retention system, according to an embodiment of the present invention.

FIG. 9 illustrates views of an attachment for weight bearing rehab, according to an embodiment of the present invention.

FIGS. 10A and 10B illustrate perspective views of an attachment for weight bearing rehab, according to an embodiment of the present invention.

FIGS. 11A-11C illustrate perspective views of an attachment for therapy, according to an embodiment of the present invention.

FIGS. 12A-12C illustrate perspective views of an attachment for therapy, according to an embodiment of the present invention.

FIGS. 13A-13C illustrate perspective views of an attachment for therapy, according to an embodiment of the present invention.

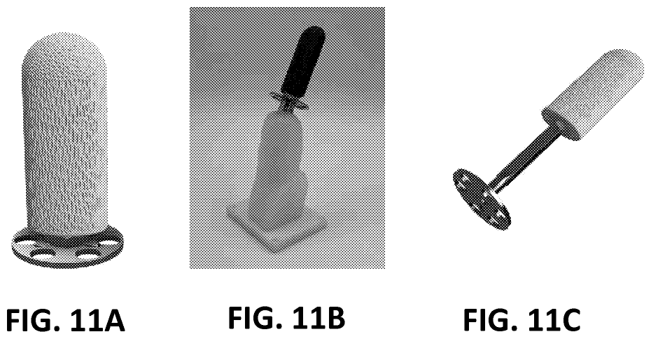

FIGS. 11A-11C illustrate perspective views of an attachment for therapy, according to an embodiment of the present invention.

FIGS. 14A-14C illustrate perspective views of a device according to an embodiment of the present invention.

FIGS. 15A and 15B illustrate perspective views of a device according to an embodiment of the present invention.

FIG. 16 illustrates views of alternate designs of a device according to the present invention.

FIG. 17 illustrates views of a design for an aesthetic finger attachment for a device according to an embodiment of the present invention.

FIG. 18 illustrates views of a design for an aesthetic finger attachment for a device according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

OI Prosthetics are composed of a silicone prosthetic, with an embedded substructure and fixture, that attaches to an abutment placed on an osseo-integrated implant. OI implants provide patients with greater functional capabilities (grasping, ADLs, osseo-proprioception, and use of technology) as well as a superior retention method. The only two providers of the implants and components for restoration (Southern Implants & Integrum) only provide a single option: rod fixture with side set screw. This basic design (metal rod) is limited in use, limited potential for customization, and has associated risks of overload to anatomy resulting in bone injury. The side set screw requires a tool and assistance/significant manual dexterity to don and doff the prosthesis.

The present invention is designed to offer patients with an alternative option for prosthetic restoration. The OI prosthetic of the present invention provide ease of donning/doffing, ease of daily hygiene, the ability to use a PT/OT program for weight training the bone, and the ability to interchange various devices for task specific applications (aesthetic finger, work finger/device, hobby device, etc.). This will provide more options for OI care teams to offer improved treatment to this patient population. Individuals that would utilize this product would include prosthetist/orthotist, Clinical anaplastologist, physical medicine and rehabilitation physicians, orthopedic surgeons, and PT/OTs.

Current standard and available designs feature a single rod and screw set retention system. The present invention allows for magnetic retention thus providing ease of donning, doffing, and hygiene. Also, the device of the present invention adds a failsafe factor: when overloading the lever arm of the system the magnet will release, preventing damage to the bone and implant. Persons missing one or more digits of the hand or foot would benefit from the present invention. Several design prototypes have been digitally created. One prototype has been beta tested and fabricated. It is currently in experimental use by a patient (quadruple amputee) for thumb restoration. Communication between the Anaplastologist/Prosthetist, PTs/OTs, Surgeons, Physicians, and Vendors prior to and post-surgery is key to successful prosthetic rehabilitation.

### Benefits of OI Prosthetics

Restores Independence
Increases consistent prosthetic retention
Improved stability provided by bone fixation
Osseoperception
   The ability to identify tactile stimuli transmitted through the osseo-integrated fixture inserted into the medullary bone canal
Increases the confidence of the patient during daily activities and allows a partial restoration of the finger functionality
Increased prosthetic use
Consistent aesthetic appearance, due to improved/secure margin integrity

### Current Downsides of OI Prosthetics

Typically, two-stage procedure for implant placement
Risk of Implant failure due to trauma, infection, and bone integrity
Granulation tissue formation around the transcutaneous component.
   The present invention works to overcome these current downsides to OI prosthetics.

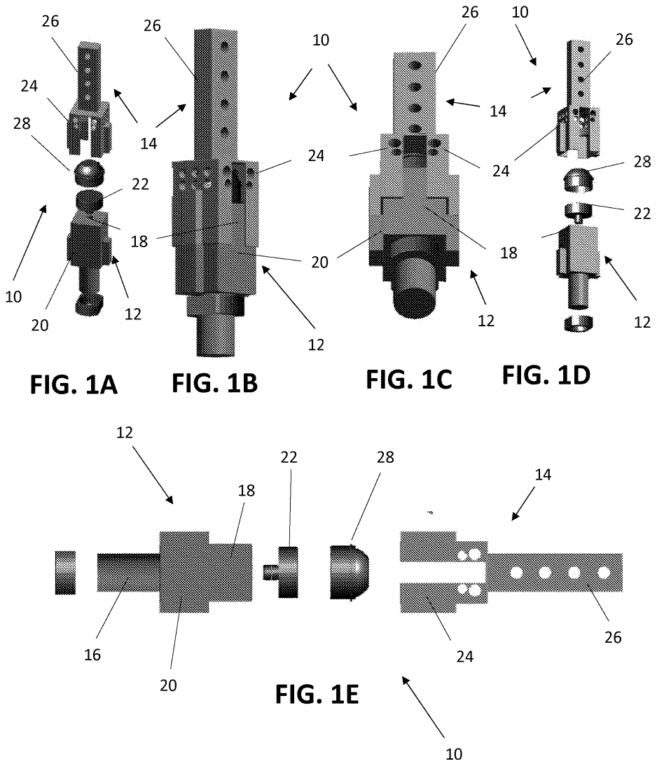

FIGS. 1A-1E illustrate perspective and partially exploded views of retention systems and abutments for an OI prosthesis, according to an embodiment of the present invention. The device 10 of the present invention includes an OI fixture 12 and an attachment component 14. The OI fixture 12 includes a threaded shaft 16 for connection to a bone and a distally positioned rectangular shaft 18. An anti-rotation wing 20 is positioned between the threaded shaft 16 and the rectangular shaft 18. A magnet 22 is disposed at a distal end of the OI fixture 12 distal to the rectangular shaft 18. The attachment component 14 includes a base 24 and a rectangular flexion shaft 26 positioned distally to the base 24. The base 24 can include perforations for coupling of a magna cap 28 and for substructure. The magna cap 28 or magnet that is used to secure the attachment component 14 to the magnet 22 of the OI fixture 12 can be cemented to the attachment component 14 with acrylic cement. The structure of the anti-rotation wing 20 in conjunction with the structure of the base 24 prevent torsional rotation of the attachment component 14 with respect to the OI fixture 12.

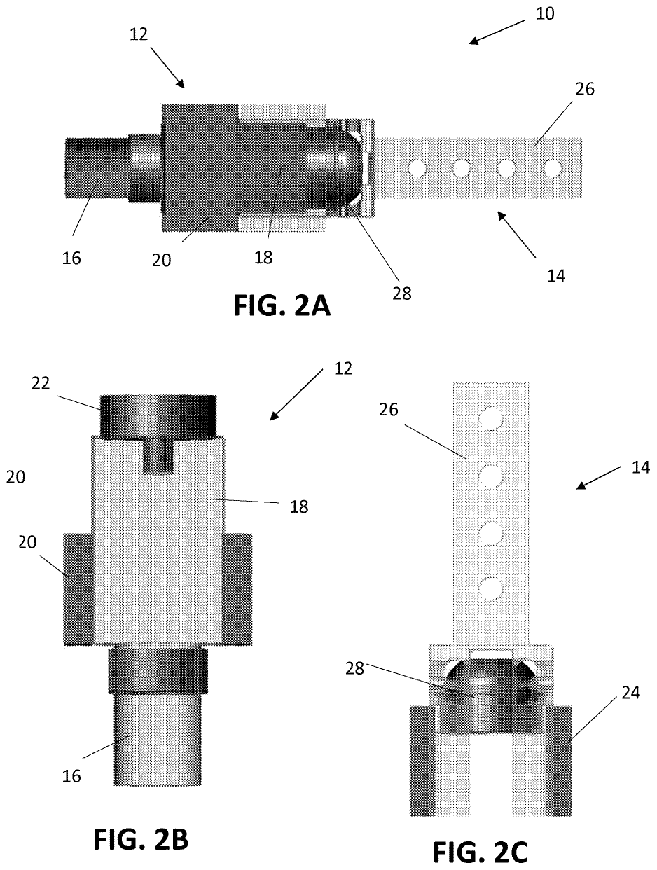

FIGS. 2A-2C illustrate side views of a retention system and abutment for an OI prosthesis, according to an embodiment of the present invention. As described above, the device 10 of the present invention includes the OI fixture 12 and the attachment component 14. The OI fixture 12 includes threaded shaft 16 for connection to bone and a distally positioned rectangular shaft 18. The anti-rotation wing 20 is positioned between the threaded shaft 16 and the rectangular shaft 18. The magnet 22 is disposed at a distal end of the OI fixture 12 distal to the rectangular shaft 18. The attachment component 14 includes the base 24 and the rectangular flexion shaft 26 positioned distally to the base. The base 24 can include perforations for coupling of a magna cap 28 and for substructure. The magnetic magna cap or magnet 28 that is used to secure the attachment component 14 to the magnet 22 of the OI fixture 12 can be cemented to the attachment component 14 with acrylic cement. The magna cap 28 can take the form of a complementary magnet or a magnetic material that will couple well with the magnet of the OI fixture 12. The structure of the anti-rotation wing 20 in conjunction with the structure of the base 24 prevent torsional rotation of the attachment component with respect to the OI fixture 12.

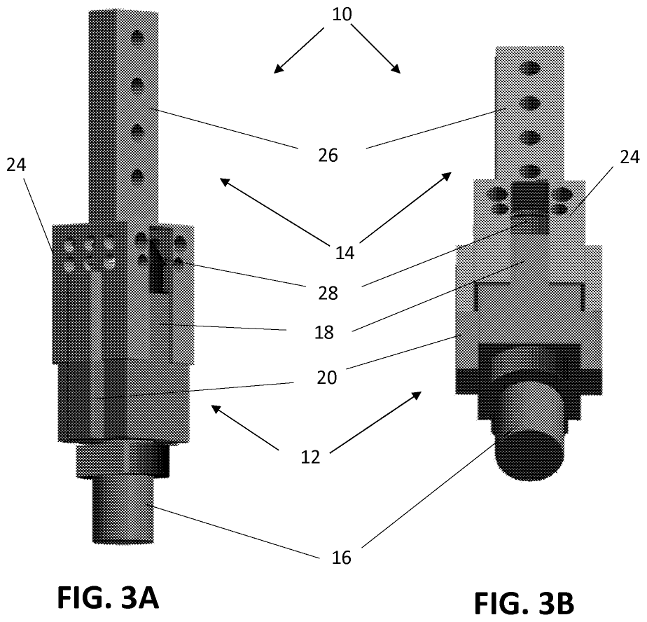

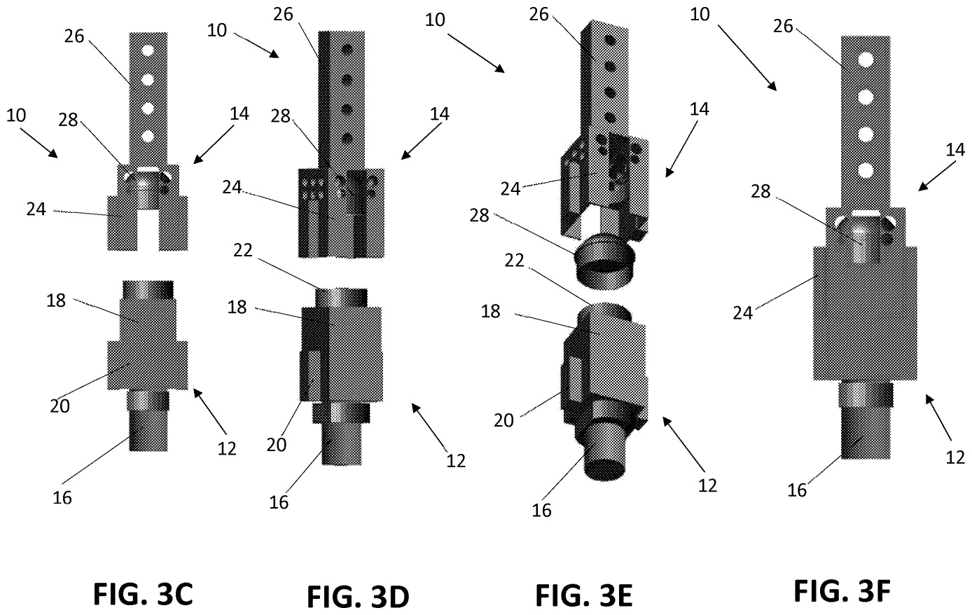

FIGS. 3A-3I illustrate perspective and partially exploded views of retention systems and abutments for an OI prosthesis, according to an embodiment of the present invention. The device 10 of the present invention includes the OI fixture 12 and the attachment component 14. The OI fixture 12 includes the threaded shaft 16 for connection to bone and a distally positioned rectangular shaft 18. The anti-rotation wing 20 is positioned between the threaded shaft 16 and the rectangular shaft 18. A magnet 22 is disposed at a distal end of the OI fixture 12 distal to the rectangular shaft 18. The attachment component 14 includes the base 24 and a rectangular flexion shaft 26 positioned distally to the base. The base 24 can include perforations for coupling of a magna cap and for substructure. The base 24 can also include structure to complement the shape of the anti-rotation wing 20, in order to further prevent rotation of the attachment component 14 relative to the OI fixture 12. The magna cap 28 or magnet that is used to secure the attachment component to the magnet 22 of the OI fixture 12 can be cemented to the attachment component 14 with acrylic cement. The structure of the anti-rotation wing 20 in conjunction with the structure of the base 24 prevent torsional rotation of the attachment component 14 with respect to the OI fixture 12.

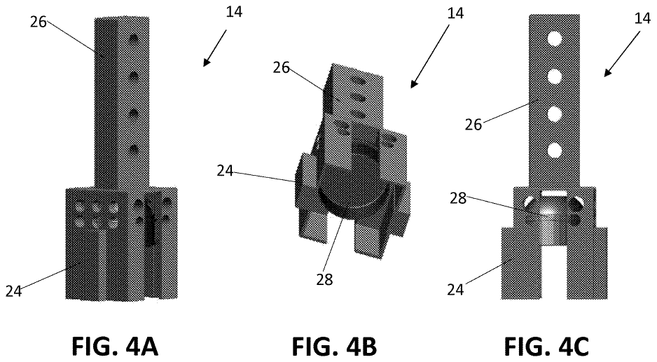

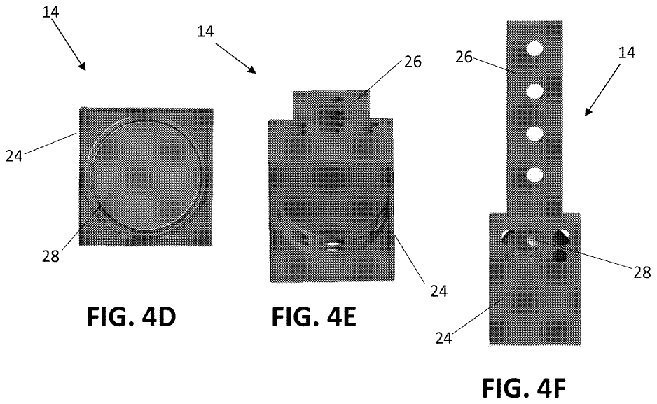

FIGS. 4A-4C illustrate perspective views of a magnetic cap insertion according to an embodiment of the present invention. The attachment component 14 includes a base 24 and a rectangular flexion shaft 26 positioned distally to the base 24. The base 24 can include perforations for coupling of a magna cap 28 and for substructure. The magna cap 28 or magnet that is used to secure the attachment component to the magnet 22 of the OI fixture 12 can be cemented to the attachment component 14 with acrylic cement. The structure of the anti-rotation wing 20 in conjunction with the structure of the base 24 prevent torsional rotation of the attachment component 14 with respect to the OI fixture 12.

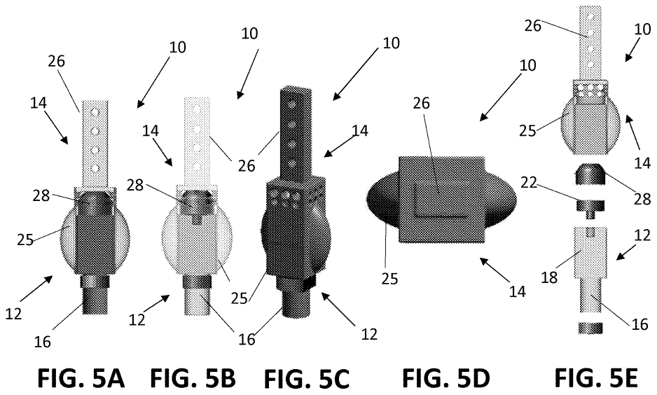

FIGS. 5A-5E illustrate perspective and partially exploded views of retention systems and abutments for an OI prosthesis, according to an embodiment of the present invention. The device 10 of the present invention includes an OI fixture 12 and an attachment component 14. The OI fixture 12 includes a threaded shaft 16 for connection to a bone and a distally positioned rectangular shaft 18. The attachment component 14 includes a base 24 and a rectangular flexion shaft 26 positioned distally to the base. The base can include perforations for coupling of a magna cap 28 and for substructure. The magna cap 28 or magnet that is used to secure the attachment component to the magnet 22 of the OI fixture 12 can be cemented to the attachment component with acrylic cement. The attachment component can include the anti-rotational wing 25. The structure of the anti-rotation wing 25 in conjunction with the structure of the OI fixture 12 and the attachment component 14 prevent torsional rotation of the attachment component 14 with respect to the OI fixture 12. In the embodiment of FIGS. 5A-5E, the base of the attachment component includes the wings 25. The extended wings 25 help to prevent rotation.

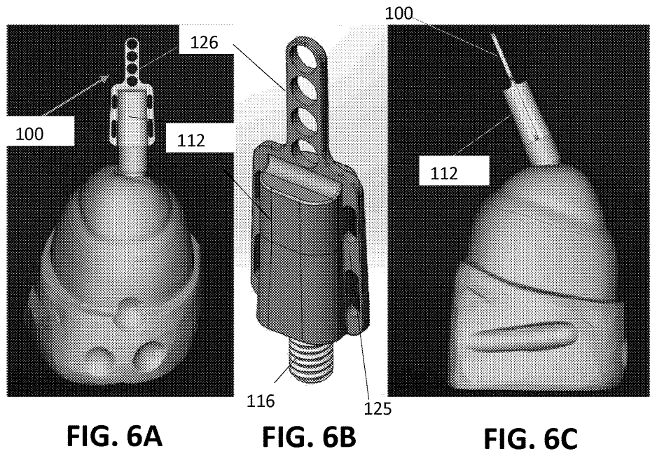

FIGS. 6A-6C illustrate perspective views of a device according to the present invention mounted on an osseo-integrated base. The device 100 of the present invention includes an OI fixture 112 coupled to a bone at a prosthesis site. and an attachment component 114 that is couplable to the OI fixture 112. The OI fixture 112 includes a threaded shaft 116 for connection to the bone and a distally positioned attachment shaft 126. An anti-rotation wing 125 is positioned between the threaded shaft 116 and the attachment shaft 126. The structure of the anti-rotation wing 125 in conjunction with the structure of the OI fixture 112 prevent torsional rotation of the attachment component 114 with respect to the OI fixture 112. In the embodiment of FIGS. 6A-6C, the base of the attachment component includes extended wings. The extended wings also help to prevent rotation.

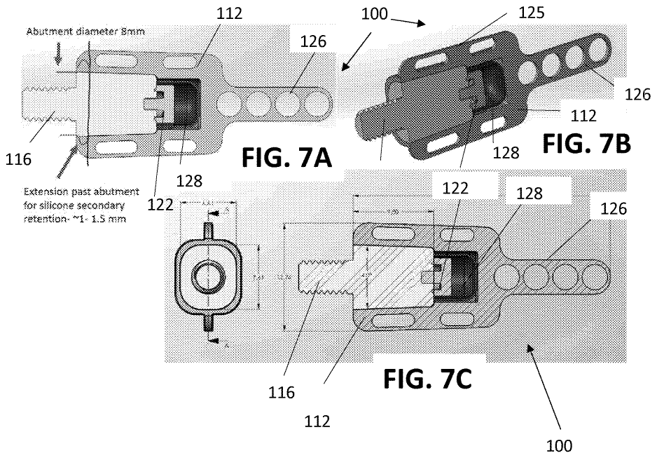

FIGS. 7A-7C illustrate perspective and semi-sectional views of an abutment and retention system, according to an embodiment of the present invention. The device 100 of the present invention includes an OI fixture 112 coupled to a bone at a prosthesis site. and an attachment component 114 that is couplable to the OI fixture 112. The OI fixture 112 includes a threaded shaft 116 for connection to the bone and a distally positioned attachment shaft 126. An anti-rotation wing 125 is positioned between the threaded shaft 116 and the attachment shaft 126. The structure of the anti-rotation wing 125 in conjunction with the structure of the OI fixture 112 torsional rotation of the attachment component 114 with respect to the OI fixture 112. In the embodiment of FIGS. 6A-6C, the base of the attachment component includes extended wings. The wings also help to prevent rotation. The magnet and the magna cap are positioned within the device, as described above.

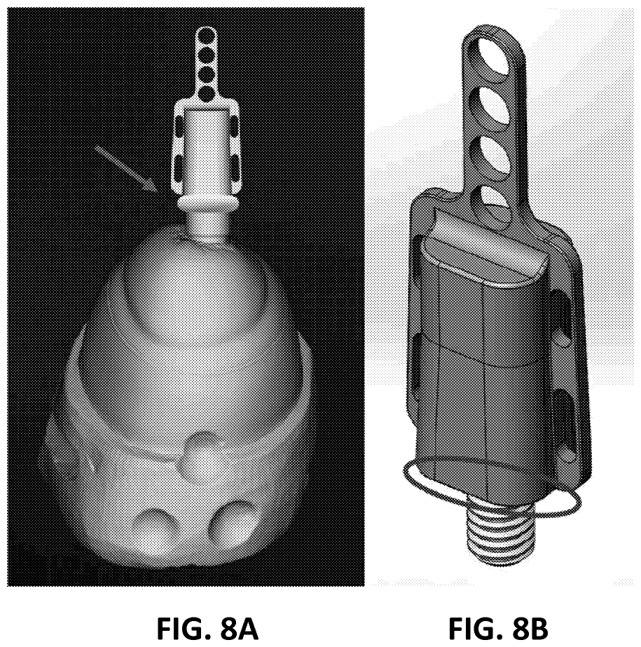

FIGS. 8A and 8B illustrate perspective views of an abutment and retention system, according to an embodiment of the present invention. The device of the present invention includes an OI fixture coupled to a bone at a prosthesis site. and an attachment component. The OI fixture includes a threaded shaft for connection to the bone and a distally positioned rectangular shaft. An anti-rotation wing is positioned between the threaded shaft and the rectangular shaft. The structure of the anti-rotation wing in conjunction with the structure of the base prevent torsional rotation of the attachment component with respect to the OI fixture. In the embodiment of FIGS. 8A and 8B, the base of the attachment component includes extended wings. The extended wings also help to prevent rotation.

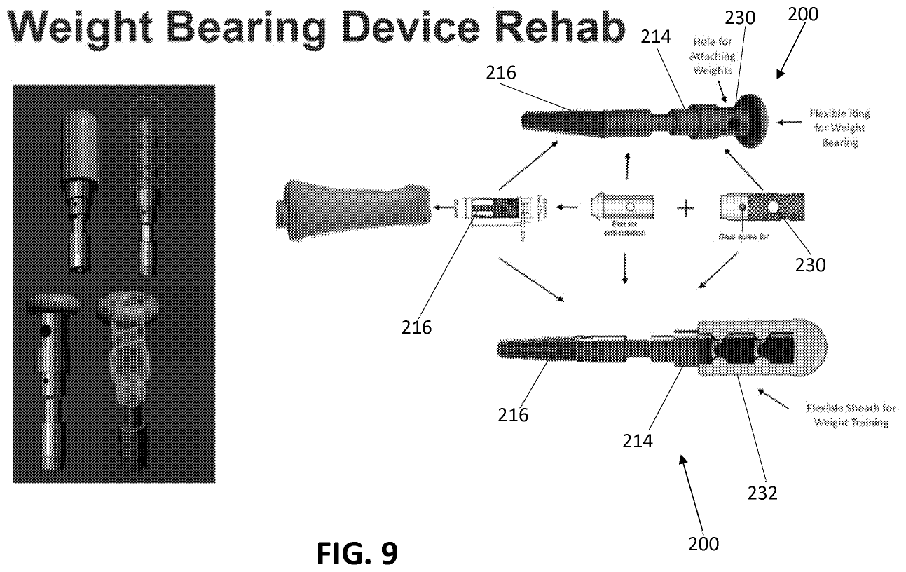

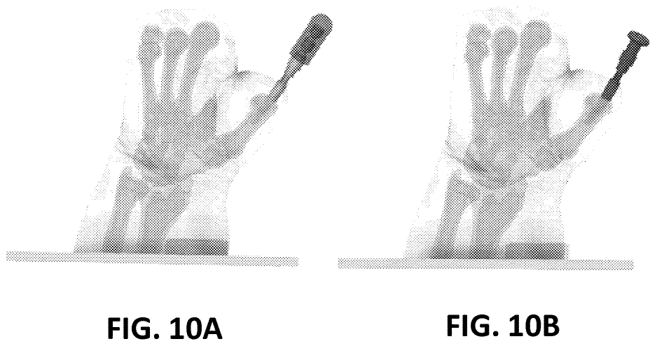

FIG. 9 illustrates views of an attachment for weight bearing rehab, according to an embodiment of the present invention. FIGS. 10A and 10B illustrate perspective views of an attachment for weight bearing rehab, according to an embodiment of the present invention. FIGS. 9, 10A and 10B illustrate a variation of the device 200 where a weight bearing rehabilitation attachment can be attached to the OI fixture 212. The weight bearing attachment can be attached for example through a hole 230 in the attachment portion. A flexible sheath 232 can also be positioned over the attachment component 214.

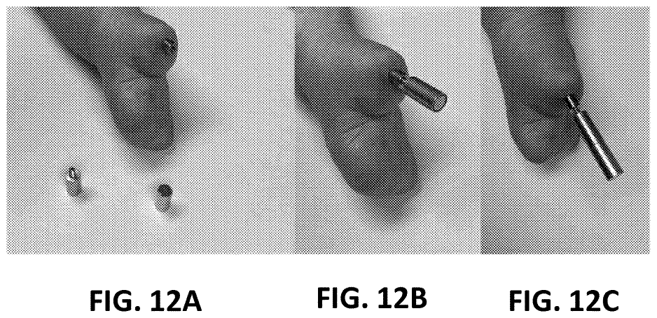

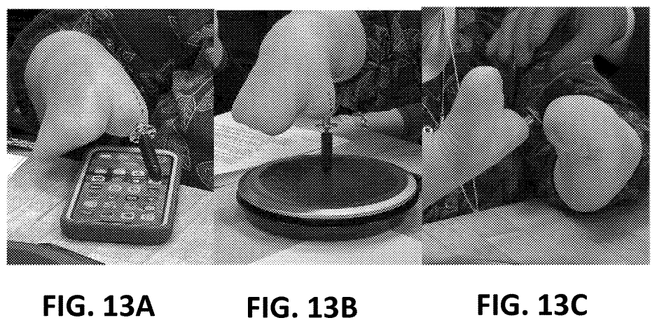

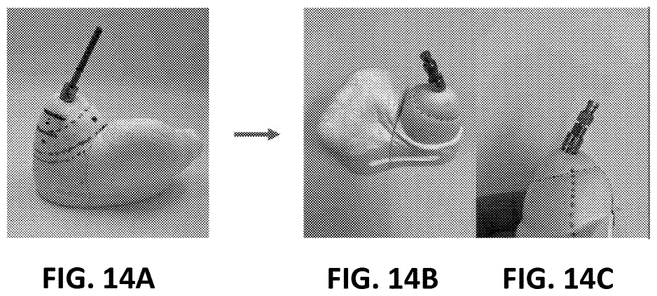

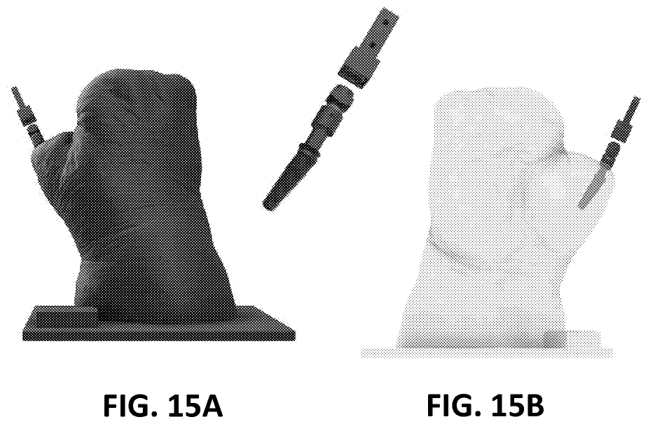

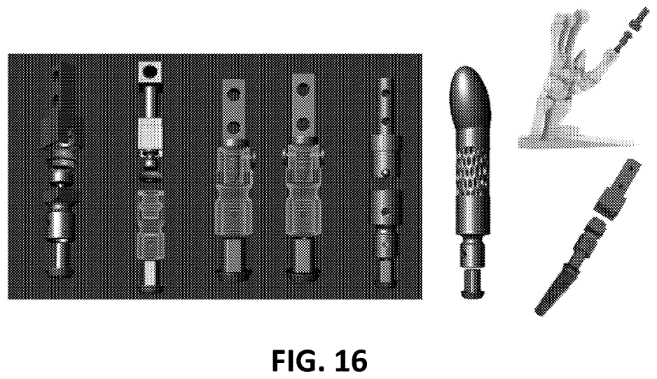

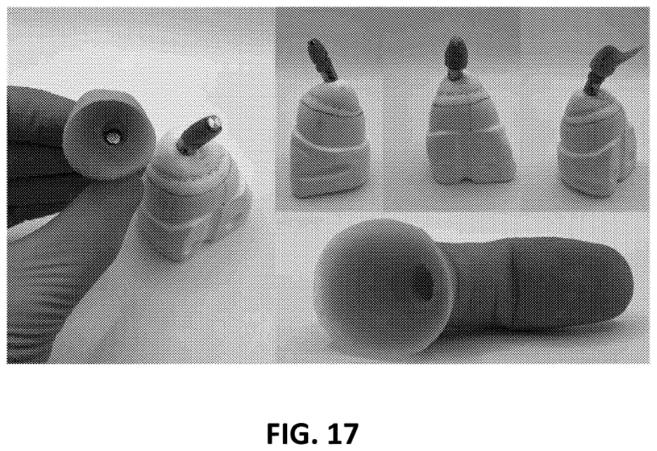

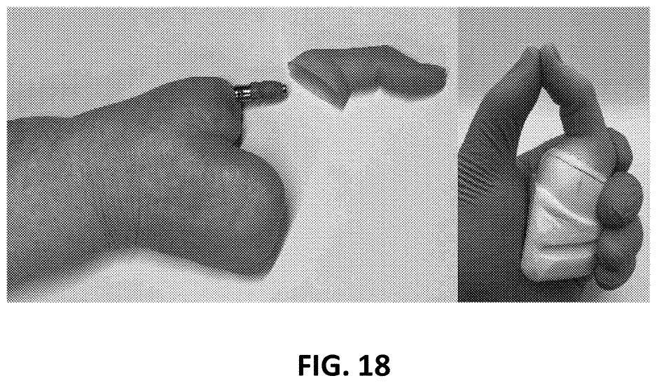

FIGS. 11A-11C illustrate perspective views of an attachment for therapy, according to an embodiment of the present invention. FIGS. 12A-12C illustrate perspective views of an attachment for therapy, according to an embodiment of the present invention. FIGS. 13A-13C illustrate perspective views of an attachment for therapy, according to an embodiment of the present invention. These interchangeable attachments for the device can be used for tasks such as working with touchscreens and other work that requires attachments for specific tasks.

FIGS. 14A-14C illustrate perspective views of a device according to an embodiment of the present invention. FIGS. 15A and 15B illustrate perspective views of a device according to an embodiment of the present invention. FIG. 14A shows a prior version of an OI prosthesis device, as compared to FIGS. 14B, 14C, 15A and 15B, which show the present invention with its exposed attachment component.

FIG. 16 illustrates views of alternate designs of a device according to the present invention. FIG. 16 illustrates various embodiments of the attachment component. These variations show other possible embodiments of the device and further illustrate that any such embodiment known to or conceivable to one of skill in the art is included herein.

FIG. 17 illustrates views of a design for an aesthetic finger attachment for a device according to an embodiment of the present invention. FIG. 18 illustrates views of a design for an aesthetic finger attachment for a device according to an embodiment of the present invention. FIGS. 17 and 18 illustrate the present invention with an aesthetic prosthetic finger attachment. As shown in FIG. 17 the aesthetic finger can be coupled to the OI fixture with a magnetic coupling. FIG. 18 shows the opposability of the aesthetic prosthetic thumb.

Applicants note that while the present invention is described herein with respect to prosthetic fingers, the present invention includes other prosthetic appendages that could be coupled to bone and designed in a similar manner with a magnetic coupling attachment component. For example, the present invention would naturally be extended to prosthetic toes and other fingers beyond the thumb.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A device comprising:
An osseo-integrated fixture, wherein the osseo-integrated fixture has a proximal end and a distal end, wherein the proximal end is configured to be integrated into a bone, and wherein the distal end includes a magnet;
an attachment component wherein the attachment component is configured to connect magnetically with the osseo-integrated fixture.

2. The device of claim 1, wherein the device is configured to provide an osseo-integrated toe.

3. The device of claim 2, wherein the attachment further comprises an aesthetic toe.

4. The device of claim 1, wherein the device is configured to provide an osseo-integrated finger.

5. The device of claim 4, wherein the attachment component comprises one selected from a group consisting of an aesthetic finger, a work finger, work device, hobby device, and a physical therapy device.

6. The device of claim 1 wherein the attachment component is configured to be coupled to a suite of tools for the user.

7. The device of claim 6 wherein the suite of tools comprises one selected from a group consisting of an aesthetic finger, a work finger, work device, hobby device, and a physical therapy device.

8. The device of claim 1 further comprising an anti-rotational wing.

9. The device of claim 1 wherein the attachment component further comprises perforations to reduce the weight of the attachment component.

10. The device of claim 1 wherein the attachment component further comprises an osseo-integrated ear.

11. A device comprising:
an osseo-integrated fixture, wherein the osseo-integrated fixture has a proximal end and a distal end, wherein the proximal end is configured to be integrated into a bone;
an attachment component wherein the attachment component is configured to connect magnetically with the osseo-integrated fixture; and,
a magnetic component for coupling the osseo-integrated fixture with the attachment component.

12. The device of claim 11, wherein the device is configured to provide an osseo-integrated toe.

13. The device of claim 12, wherein the attachment further comprises an aesthetic toe.

14. The device of claim 11, wherein the device is configured to provide an osseo-integrated finger.

15. The device of claim 14, wherein the attachment component comprises one selected from a group consisting of an aesthetic finger, a work finger, work device, hobby device, and a physical therapy device.

16. The device of claim 11 wherein the attachment component is configured to be coupled to a suite of tools for the user.

17. The device of claim 16 wherein the suite of tools comprises one selected from a group consisting of an aesthetic finger, a work finger, work device, hobby device, and a physical therapy device.

18. The device of claim 11 further comprising an anti-rotational wing.

19. The device of claim 11 wherein the attachment component further comprises perforations to reduce the weight of the attachment component.

20. The device of claim 11 wherein the attachment component comprises an osseo-integrated ear.
